(19) United States
(12) Patent Application Publication
(10) Pub. No.: US 2006/0246574 A1
(43) Pub. Date: Nov. 2, 2006

(54) Dispenser for making a lateral flow device

(76) Inventors: Sarah Rosenstein, Evanston, IL (US); Craig A. Olbrich, Corvallis, OR (US); John S. Dunfield, Corvallis, OR (US); Lauren Renee Henry, Dallas, OR (US); Christie Lee Dudenhoefer, Corvallis, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/117,848

(22) Filed: Apr. 29, 2005

(51) Int. Cl. C12M 1/34

(52) U.S. Cl. 435/287.2

(57) ABSTRACT

A dispenser for building a lateral flow immunoassay device comprises a fluid supply assembly and a jetting assembly. The fluid supply assembly comprises an antibody component including a plurality of reservoirs to supply at least one of member of a group comprising a species array of at least one species-specific antibody material, an anti-species array of at least one first anti-species antibody material, and a label-antibody array of at least one of a tag material and a second anti-species antibody material. The jetting assembly is in communication with the fluid supply assembly to enable dispensing the materials of the species array, the anti-species array, and the label-antibody array from the supply assembly onto a substrate.

### CROSS-REFERENCE TO RELATED APPLICATIONS

This application is related to U.S. Patent Application No. ______, titled LATERAL FLOW DEVICE filed on even date herewith, and which is incorporated herein by reference.

### BACKGROUND

Immunoassay testing is a promising technology for testing a wide variety of biological elements. One form of immunoassay testing includes immunochromatographic test strips, sometimes known as lateral flow through (LFT) sensors. For example, conventional LFT sensors include tests in which a color band appears (or is absent) when a specific antigen is present in a human fluid, such as detecting human chorionic gonadotropin (hCG) in urine for pregnancy testing. These sensors enable home testing or convenient clinic-based testing of common biological conditions.

However, many tests reveal false positive or false negatives due to a variety of factors, including a user's inability to discern the color changed revealed by the LFT sensor, as well as misunderstanding or misremembering whether the presence or absence of a stripe on the LFT test strip reveals a positive result or a negative result. Coupled with imperfections in manufacturing LFT test strips and challenges in producing easily identifiable results, these human-operator errors make use of these LFT test strips less than ideal, despite their convenience. Moreover, the results of the tests fade over time, thereby providing only a temporary record of the test. In addition, the presence or absence of a stripe on a LFT sensor provides only a qualitative result for the biologic condition being tested. Finally, conventional manufacture of LFT test strips includes dipping or spraying biological substances onto a substrate, which requires relatively large volumes of these biological substances and bulky assembly equipment. Moreover, other smaller volume dispensing devices have had little impact on the basic manner of conventional manufacture of lateral flow devices.

With these challenges, the full benefit of lateral flow through (LFT) testing has not been fulfilled.

### SUMMARY

Embodiments of the invention are directed to lateral flow devices and related systems and methods. One embodiment of the invention is directed to a dispenser for building a lateral flow immunoassay device comprising a fluid supply assembly, and a jetting assembly. The fluid supply assembly comprises an antibody component including a plurality of reservoirs to supply at least one of member of a group comprising a species array of at least one species-specific antibody material, an anti-species array of at least one first anti-species antibody material, and a label-antibody array of at least one of a tag material and a second anti-species antibody material. The jetting assembly is in communication with the fluid supply assembly to enable dispensing the materials of the species array, the anti-species array, and the label-antibody array from the supply assembly onto a substrate.

### BRIEF DESCRIPTION OF THE DRAWINGS

FIG. 1 is a block diagram of a dispensing system, according to an embodiment of the present invention.

FIG. 2 is a block diagram of a dispensing station, according to an embodiment of the present invention.

FIG. 3 is a plan view schematically depicting a lateral flow device, according to an embodiment of the present invention.

FIG. 4 is a sectional view of a lateral flow device, according to an embodiment of the present invention.

FIG. 5 is a plan view schematically depicting a lateral flow device, according to an embodiment of the present invention.

FIG. 6A is block diagram of a digital imager system, according to an embodiment of the present invention.

FIG. 6B is chart illustrating a time rate of change of color over time for a lateral flow device, according to an embodiment of the present invention.

FIG. 7A is an enlarged plan view of a results portion of a lateral flow device, according to an embodiment of the present invention.

FIG. 7B is an enlarged plan view of a results portion of a lateral flow device, according to an embodiment of the present invention.

FIG. 8 is a plan view of a lateral flow device with multiple lateral flow modules, according to an embodiment of the present invention.

FIG. 9 is a partial plan view of a lateral flow device with portions exposed to schematically illustrate a capillary structure, according to an embodiment of the present invention.

FIG. 10 is a block diagram of a control monitor, according to an embodiment of the present invention.

### DETAILED DESCRIPTION

In the following detailed description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

Embodiments of the present invention are directed to lateral flow devices for performing immunoassay testing, and methods of making lateral flow devices. These embodiments enable highly accurate testing of one or more analytes on a single test strip, in either a human readable or a machine-readable format. Analytes are identified by interactions of an antigen of the analyte molecules with corresponding anti-bodies for the antigen.

In one embodiment, a dispensing system is configured for dispensing liquids and/or powders, such as biological substances used in immunoassay testing, particularly lateral flow through (LFT) sensors. Biological substances (herein “biosubstances”) includes biological flowable materials which includes, but is not limited to, human, plant, and/or animal fluids, such as urine, blood, saliva, etc. that are suitable for use in immunoassay testing. Biosubstances also includes components and subcomponents of human, animal, plant fluid, such as various molecules, including but not limited to components to which an immunological response can be mounted via an antibody. Accordingly, these components and subcomponents comprise proteins, electrolytes, hormones, viruses, sugars, lipids, etc. In some embodiments, analytes of interest include DNA, RNA, oglionucleotides, for detecting genetic-related diseases and other genetic-based testing.

Additional non-limiting examples of analytes detectable with a lateral flow device of the present invention comprise a pregnancy analyte (e.g. human chorionic gonadotropin known as hCG), one or more cardiac panel markers (e.g., cortisol, low density lipoprotein (LDL), myoglobin, troponin, etc.), common elements in blood (e.g., glucose, pH, etc), drugs-of-abuse analytes (e.g., alcohol, cocaine, heroin, methamphetamine, marijuana, ecstasy, etc.), performance enhancing elements (e.g. steroids, human growth hormone, blood doping factors, etc.), viruses and bacteria (e.g. SARS, HIV, hepatitis, flu, pox, E. coli, etc), sexually transmitted diseases (STD) (e.g., syphilis, gonorrhea, etc). Ordinary chemicals and substances are also the subject of analyte testing with embodiments of the invention, including but not limited to, caffeine, sodium, etc.

In one embodiment, biosubstances also include fluid mediums which act as a carrier to enable biological materials to flow along a lateral flow device. In another embodiment, biosubstances also include materials capable of affecting biological materials, such as chemicals, pharmaceutical agents, reagents, etc. suited to facilitate the immunoassay mechanisms on the lateral flow device. In one embodiment, the biosubstances include protein-blocking agents, such as BSA, for blocking the action of binding sites for non-target antigens and/or antibodies, to thereby prevent inadvertent indication of target activity.

Immunoassays include enzyme immunoassays including but not limited to competitive assays, as well as sandwich assays, and other assays suitable for the lateral flow through (LFT) format. Immunoassays embodied in lateral flow devices of an embodiment of the invention are capable of various detection methods such as chemilluminescent detection, colorimetric detection, and fluorescent detection. In one embodiment, calorimetric detection is employed in which the optical quality or quantity of a colored sample is evaluated by human observation and/or by a machine such as a digital imager.

In one embodiment, a dispensing system includes at least one drop-on-demand jetting device, such as an inkjet printhead, for depositing biosubstances and other materials from an array of fluid modules (e.g. reservoirs) onto a test strip base to create the immunoassay with precision and accuracy. In one embodiment, the jetting device comprises a thermal inkjet (TIJ) printhead. This dispensing system enables building a LFT device with highly accurate, measured volumes of antibodies and support materials to achieve effective, reliable quantitative testing for antigens. In addition to depositing small volumes of antibodies, in some embodiments, the dispensing system is used to create layers and/or entire functional units of the immunoassay upon a fluid flow member. Other attributes of the dispensing system used in building LFT devices are described throughout the description.

In one embodiment, a lateral flow device produces a quantitative test result indicating a concentration of an analyte detected in a sample, via antigen-antibody interaction by immunoassay techniques. The quantitative test and result is provided by an arrangement, via the dispensing system, of a test capture field including one or more lines, words, symbols, and/or patterns, as well as optical parameters of a color of the test capture field. The quantitative result is observed by machine-readable mechanisms, such as a digital imager, or by indicators readable by the human eye.

In another embodiment, a lateral flow device tests for multiple analytes is built by arranging each analyte test generally parallel to each other along a length of the lateral flow device, via the dispensing system, so that the different analyte tests are performed independently from each other, in parallel, as the sample fluid flows across the lateral flow device. In one embodiment, a barrier is deposited between adjacent test lanes to maintain separation of the parallel analyte tests. In other embodiments, a membrane supporting the analyte test lanes includes capillaries that are oriented along a single direction to maintain flow of the fluids within each analyte test lane.

In one embodiment, each of the functional modules of lateral flow device, or portions thereof, are deposited as layers onto a carrier via the dispensing system. These functional modules include, but are not limited to, a reagent module, a reaction module, and a waste module. The reagent module tags an antigen molecule with a flowable particle coated with an antibody to enable the antigen to flow along the LFT device and be identified more readily. The reaction module (or results module) comprises a test module for capturing the antigen-particle complex to reveal the presence or absence of the antigen, and a control module for indicating adequate flow of the sample along the LFT device. The dispensing system enables precise location of depositing the biosubstances forming these modules, as well as precision in the volume of biosubstance(s) deposited, enabling control over the thickness of each layer, as well as exact quantities of the biological substances being deposited, whether in lines, spots, patterns, words, or symbols—with or without color. Controlling the thickness of the layer enhances calorimetric detection by enhancing optical density factors that affect machine reading of color changes in the LFT test strip.

In another embodiment, a drop-on-demand jetting device system enables multiple different biological substances to be deposited contemporaneously, or sequentially, onto a single location of one or more pixels. In one aspect, each nozzle of a plurality of nozzles (e.g., an orifice plate) of the dispensing system deposits a different biosubstance or different material onto a single location of a membrane of the LFT device to create a functionalized biological unit at that single location. This functionalized unit on a single location can be referred to as a functional biounit. In other words, several different biosubstances (or materials) that together enable a particular reaction or foundation for a reaction are deposited effectively as one in a single location on the test strip via the dispensing system.

The functional biounit can vary in size and/or volume, as selected by the operator with the various nozzles ejecting the requested amount of biosubstances or materials. Moreover, the different biosubstances can be ejected onto the functional biounit at different points in time to control reaction kinetics between the different biosubstances and materials being deposited to form the functional biounit.

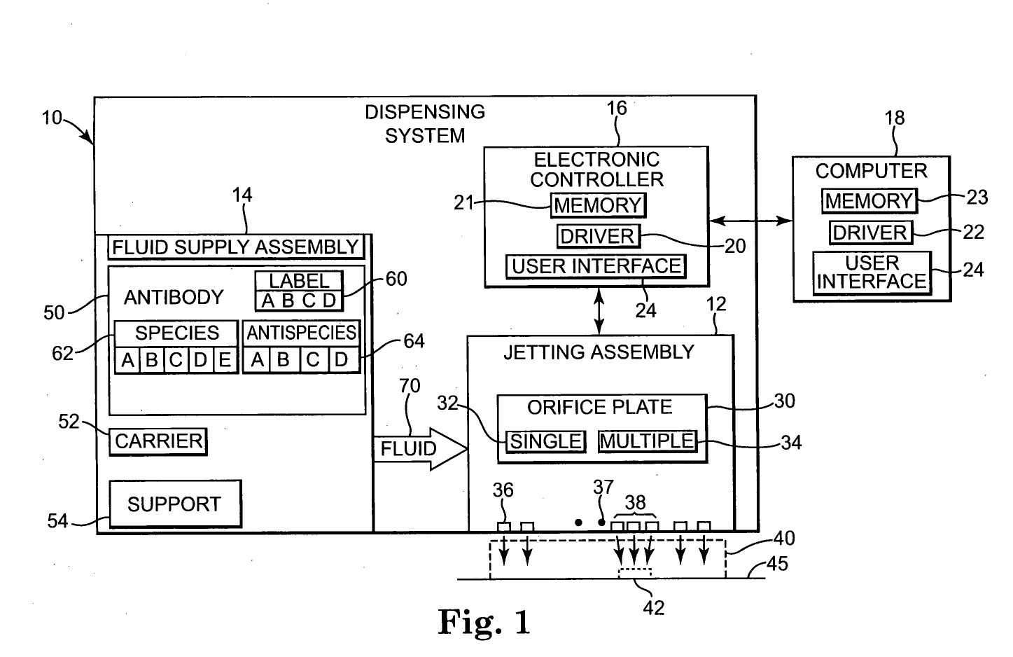

These embodiments of the invention, and additional embodiments of the invention, are described and illustrated throughout FIGS. 1-9.

FIG. 1 is a block diagram of a dispensing system of one embodiment of the invention adapted for building a lateral flow device. As shown in FIG. 1, dispensing system 10 comprises jetting assembly 12, fluid supply assembly 14, electronic controller 16. In one embodiment, computer 18 is in electrical communication with dispensing system 10 via controller 16 and includes, among other things, driver 22, memory 23, and a user interface 24.

Controller 16 comprises jetting driver 20 while computer 18 comprises jetting driver 22. In one embodiment, jetting driver 22 of computer 18 can provide all jetting driver functions without jetting driver 20 of controller 16, or act in cooperation with jetting driver 20 of controller 16. In another embodiment, jetting driver 22 of computer 18 can provide all jetting driver functions without jetting driver 20 of controller 16, or act in cooperation with jetting driver 20 of controller 16. Accordingly, functions and features described in association with jetting driver 20 will also be attributed to jetting driver 22, and vice versa. Jetting driver 20 is stored in memory 21 of electronic controller 16 while jetting driver 22 is stored in memory 23 of computer 18. In one embodiment, jetting driver 20 and/or jetting driver 22 is a printer driver.

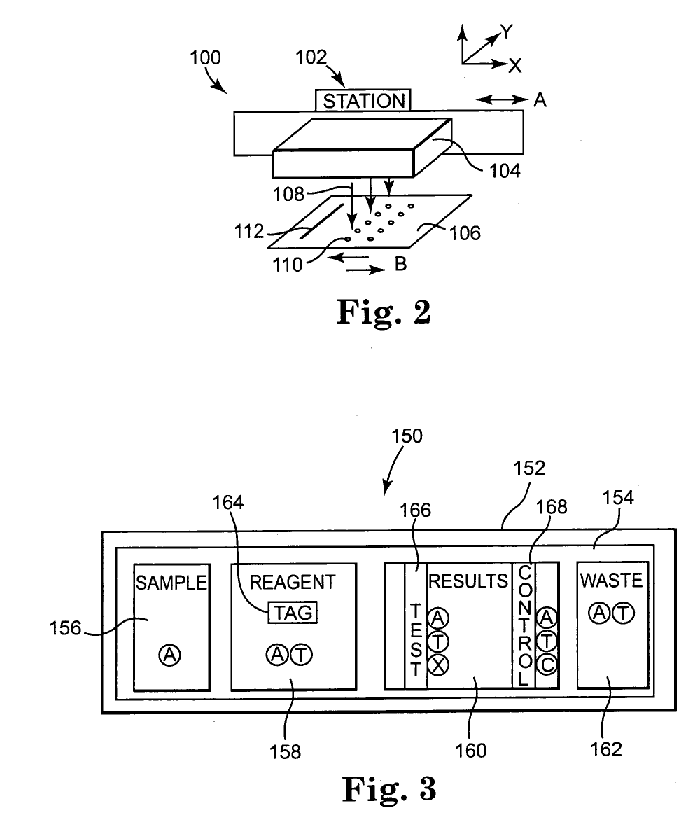

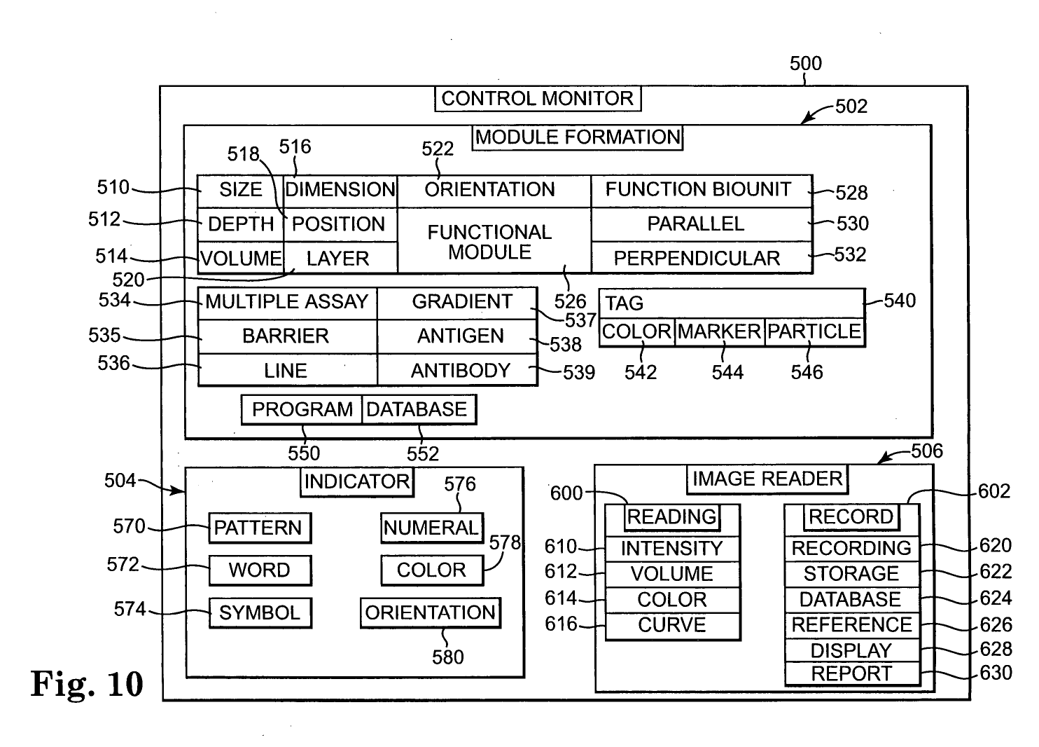

Jetting driver 20 and/or jetting driver 22 act to direct dispensing of various fluids for depositing on a substrate via jetting assembly 12. As will be explained in more detail in association with FIGS. 2-9, driver(s) 20, 22 direct specific depositing of biological substances, fluids, etc. onto a carrier to build a lateral flow device for immunoassay testing. In one embodiment, manipulation of dispensing system 10, including jetting driver 20 or 22, can be performed via a control monitor of controller 16 (via user interface 24), such as control monitor 500 as later illustrated and described in association with FIG. 10.

Jetting assembly 12 comprises, among other things, orifice plate 30 and nozzles 36 for printing fluids 37 (as drops) through jetting zone 40 onto medium 45. In one embodiment, orifice plate 30 comprises single orifice plate 32 in which all of the fluid(s) from fluid supply assembly 14 are jetted onto medium 45 through single orifice plate 32 via nozzles 36. In another embodiment, orifice plate 30 comprises multiple orifice plates 34 in which some of the fluids from fluid supply assembly 14 (e.g. species-specific antibodies) are jetted onto medium 45 via nozzles 36 of a first orifice plate and other fluids from fluid supply assembly 14 (e.g. anti-species specific antibodies) are jetted onto medium 45 via nozzles 36 of a second orifice plate separate from the first orifice plate. Separation of the multiple orifice plates 34 prevents cross-contamination of the fluids associated with one orifice plate relative to fluids associated with another separate orifice plate. In one embodiment, additional orifice plates are used so that each different fluid to be jetted is matched with its own separate orifice plate.

In one embodiment, jetting assembly 12 comprises a fluid ejection device. In one embodiment, jetting assembly 12 comprises a thermal inkjet technology (TIJ) fluid ejection device, such as a thermal inkjet printhead. One such printhead is available from Hewlett-Packard Corporation. In one aspect the fluid ejection device is a drop-on-demand fluid ejection device. In another aspect, the fluid ejection device is a piezoelectric fluid ejection device, continuous ink jet device, or other type of ejection device (e.g., acoustic) capable of jetting small volumes of fluids.

In addition, in one embodiment jetting assembly 12 comprises at least one set of nozzles 38 arranged on an orifice plate 30, and controlled via drivers 20/22, to dispense a separate fluid (or the same fluid) from each different nozzle onto the single location 42 on medium 45. This feature enables different biosubstances to be deposited together as a single functional unit (e.g., a functional biounit) into a single location 42 on medium 45, as well as, enabling a reaction of different biosubstances at a single location via direct depositing from separate nozzles (or from separate orifice plates).

Fluid supply assembly 14 comprises a plurality of reservoirs of fluid (e.g., flowable materials) including antibody fluid module 50 which includes label array 60, species array 62, and anti-species array 64. Label array 60 comprises one or more components for facilitating the flow of an antigen or antibody through a membrane of an LFT assay and/or that makes the antigen or antibody visibly detectable by human eyesight or machine reading (e.g. digital imager). In one embodiment, some aspects of label array 60 include flowable particles, such as microspheres or latex beads, which can be different colors. In another aspect, label array 60 comprises one or more metals (e.g., gold) for associating a visible indicator with an antigen and/or antibody.

Species array 62 comprises one or more antibodies specifically responsive to the binding site of the target antigen in the sample, typically known as a species-specific antibody or target-specific antibody. The species-specific antibodies typically are used for establishing a test line or test component in a lateral flow device, and therefore can be referred to as a test fluid or test biosubstance.

Anti-species array 64 comprises one or more antibodies responsive to a binding site on the antigen but that do not compete with the species-specific antibody for binding at the antigen-binding site. The anti-species antibodies are used for establishing a control line in a lateral flow device, and therefore also can be referred to as control materials or control biosubstances.

For any immunoassay tests that use a different configuration of the respective antigen and antibody binding mechanisms, fluid assembly 14 can be configured to hold those respective biosubstances for selective communication to jetting assembly 12.

Fluid supply assembly 14 is in communication with jetting assembly 12 so that fluid 70 flows into jetting assembly 12 to supply nozzles 36 with fluid 37 for jetting as separate spots, lines and/or other patterns onto medium 45. In one embodiment, fluid supply assembly 14 is separate from jetting assembly 12 while in other embodiments, fluid supply assembly 14 is integral with jetting assembly 12. Moreover, fluids in fluid supply assembly 14 are each contained within separate reservoirs together in a single housing, or contained in separate reservoirs in multiple housings. For example, in one embodiment, each of different species A, B, C, D, E of species array 62 are contained in separate reservoirs within a single housing and each label (e.g., latex beads) of label array 60 and each antispecies fluid of antispecies array 64 are each contained within their own housings. In another embodiment, both label array 60 and anti-species array 64, while contained within separate reservoirs are located within a single housing that cooperates with jetting assembly 12.

Carrier module 52 of fluid supply assembly 12 comprises one or more fluids used to carry some other active or passive component along the lateral flow assay. Support module 54 of fluid supply assembly 12 comprises one or more chemically active or biologically active substances used for causing, inhibiting, or encouraging appropriate flow, migration, or reactions among assay components for various purposes (e.g., affecting reaction kinetics of the immunoassay). Support fluids or substances, include but are not limited to, surfactants, adjuvants, as well as protein blockers, screening materials, etc. In some embodiments, support module 54 also includes substances that are inert or non-reactive with the antigen (or with the antibodies), but that otherwise enable reaction mechanisms and/or binding mechanisms to occur on a lateral flow device. In one embodiment, such support fluids include sucrose or hydro-sensitive materials or hydro-responsive materials (e.g., hydrophobic or hydrophilic polymers) that enhance directed flow of microspheres along a fluid flow member or membrane.

Finally, embodiments of the present invention are not strictly limited to the configuration of the fluid supply assembly 14 and jetting assembly 12 as illustrated in FIG. 1, but extend to other combinations of fluid reservoirs, fluid supply assemblies, jetting devices, and jetting assemblies, in separate or integral cartridge forms.

In one embodiment, the jetting assembly 12 comprises a thermal inkjet (TIJ) fluid ejection device, such as a thermal inkjet printhead available from Hewlett-Packard of Palo Alto, Calif. The ejection device comprises an orifice plate having nozzles on the order of 10 microns, although larger nozzle sizes such as 50 microns, 100 microns, 500 microns, and 1000 microns (and all values in-between these examples). In cooperation with an appropriately sized nozzle, a firing chamber of the ejection device is configured to dispense a single drop on the order of 5 to 10 picoliters, as well as larger volumes such as 1 or 10 nanoliters. When larger volumes of biosubstances are to be dispensed, even larger nozzles and/or firing chambers can be used. However, the smallest sized nozzles and firing chambers described are desirable for their precision in dispensing very small volumes of biosubstances, which facilitates building and use of a lateral flow device, as well as reading the results of the lateral flow device. In one embodiment, drops are dispensed on the order of 1-100 microns, as well as larger drops when desired. By use of a positioning mechanism, the drops can be dispensed in desired patterns, lines, symbols, etc., onto the lateral flow device.

These volumes and sizes of drops ejected by the jetting device are determined by several parameters of the fluid ejection device. These parameters include but are not limited to, the size of the thermal element, the size of the orifice nozzles, as well as the volume of the firing chamber. Accordingly, a fluid ejection device can be selected or constructed with these parameters to yield a jetting assembly especially adapted to dispense biosubstances to create a lateral flow device for the embodiments of the invention illustrated and described in association with FIGS. 1-9.

Finally, because of the limited time of exposure in the thermal inkjet printhead, the biosubstances are not substantially affected despite the very high temperatures generated by the thermal element. Accordingly, the thermal inkjet printhead provides a highly accurate dispensing mechanism for dispensing quantifiable volumes of biosubstances while not comprising the biologic activity of the dispensed biosubstances.

FIG. 2 illustrates one embodiment of a positionable dispensing system 100. As shown in FIG. 2, positionable dispensing system 100 comprises positioning station 102, dispenser 104, and target media 106. Dispenser 104 has substantially the same features and attributes as dispensing system 10 of FIG. 1. Dispenser 104 deposits drops 108 onto target media 106 as spots 110, lines 112 or other shapes and patterns. In one embodiment, drops are dispensed by dispenser 104 onto target media 106 while target media 106 is moved, as represented by directional arrow B, relative to dispenser 104 which is held stationary by positioning station 102. In another embodiment, drops are dispensed by dispenser 104 onto target media 106 while dispenser 104 is moved relative to target media 106 via positioning station 102, as represented by directional arrow A. Moreover, positioning station 102 can position dispenser 104 into a fixed position over target media 106 along any one or more of three axes x, y, z.

In one embodiment, positionable dispensing system 100 is also configured to cause rapid relative movement between a series of target media 106, such as membranes of lateral flow through devices, and dispenser 104 to enable depositing biosubstances onto the target media 106, thereby enabling large quantities of lateral flow devices to be built according to embodiments of the invention.

FIG. 3 is a plan view schematically depicting a lateral flow device 150, according to one embodiment of the invention. As shown in FIG. 3, device 150 comprises carrier 152, membrane 154, sample module 156, reagent module 158, results module 160, and waste module 162.

Carrier 152 comprises a plastic or other semi-rigid material for supporting the other elements of the immunoassay test. Membrane 154 comprises a fluid flow member configured to induce or enable flow of fluid along a length of the lateral flow device. In one embodiment, membrane 154 comprises a nitrocellulose fiber pad or matrix. In other embodiments, membrane 154 comprises a cellulose acetate membrane or a glass fiber membrane. The size, porosity, and type of membrane 154, is selected along with the size, and type of labels (e.g., flowable particle, microspheres, and beads), and volume and types of fluids and biosubstances of the lateral flow device 150 to achieve desired reaction kinetics of the immunoassay. These reaction kinetics are further based on flow rates, reaction rates, assay time, reagent usage, dimensions of the components of the lateral flow device, flow capacity of the membrane, and binding capacity of the membrane, etc.

Sample module 156 comprises a portion of a capture pad for receiving a sample fluid, such as saliva, blood, urine, or other body fluids, that contains an antigen molecule (A). In some embodiments, the sample is diluted or otherwise modified in preparation for the immunoassay test provided by lateral flow device, either to improve migration of the antigen molecule (A) along the lateral flow device 150 or to improve reaction properties of the antigen molecule (A) with the various biological and/or chemical components along the lateral flow device 150. Sample module comprises a screening component made of a physical device, biological materials, and/or chemical materials adapted to separate out larger particulates and other components of a sample fluid to prevent interference of these particulates or interferents with intended reaction mechanisms of the immunoassay test.

Reagent module 158 of lateral flow device 150 is loaded with various components for equipping the sample material, e.g., an antigen molecule (A), to flow through membrane 154 and to interact with appropriate antibody components in results module 160 as the antigen molecule travels along the remainder of the lateral flow device 150. In one embodiment, reagent module 158 comprises a tag 164 embedded within reagent module 158, which is a flowable particle (e.g., a microsphere or latex bead) that attaches to the antigen molecules, via an antibody coated on the particle, to enable the antigen molecule to flow through membrane 154 along lateral flow device 150. In this aspect, binding of the antibody of the tag 164 to the antigen molecule will not indicate a negative or positive test result in results module 160 since the antibody of tag 164 binds with a site on the antigen molecule different than the site of the antigen molecule for which the test is being performed. Accordingly, the antibody of the tag is used merely for attaching the tag 164 to the antigen molecule.

Once the antigen molecule is marked with tag 164 via reagent module, it is referred as a tagged antigen particle (illustrated as AT in FIG. 3), which is understood to refer to the entire complex of the antigen molecule, flowable particle, and color marker (if present).

In one embodiment, each tag 164 comprises a colored flowable particle. The color is provided by colored latex beads or by attaching a colored metal to the flowable particle. In another embodiment, in which a sample includes different antigen molecules that are being detected, different colored tags 164 are present in reagent module 158 with each different colored tag 164 being configured with an antibody for binding with the antigen molecule corresponding to the color intended to be represented by that color tag 164. Accordingly, each different color tag 164 typically uses a unique antibody, different from the antibody of the other color tags 164, for attaching to its target antigen molecule.

Finally, in one embodiment, each of the biosubstances and/or materials comprising reagent module 158, including but not limited to, tags 164 with their accompanying flowable particles, antibody, and/or color, is printable onto membrane 154 via jetting assembly (see FIG. 1 and accompanying text) in substantially the same manner as previously described in association with FIGS. 1-2.

As shown in FIG. 3, results module 160 of lateral flow device 150 comprises at least one test component 166 and at least one control component 168. In one embodiment, the test component 166 is an immobile pattern (e.g., a target line) extending laterally across the results module 160) of a species-specific antibody adapted for binding with an available site on the tagged antigen particle (AT) in the sample.

In a competitive assay test, when the target antigen is present in the sample, the tagged antigen particle (AT) that is traveling along the lateral flow device 150 becomes bound to the available species-specific antibody in the test component 166 of results module 160, thereby becoming fixed to the test component 166. Additional tagged antigen particles (AT) flowing along lateral flow device 150 are also immobilized by the target line of test component 166 (illustrated as the complex ATX), so that when a significant number of tagged antigen particles are captured, target line is readily visible by virtue of aggregation of the color element in the tagged antigen particles at test component 166.

In one embodiment, results module 160 comprises a test component 166 including cardiac panel markers, which typically includes several different antigen molecules that function separately but collectively indicate a cardiac condition of a patient. In one aspect, three cardiac panel marker antigens will be available in the sample as antigens to be detected. Accordingly, test component 166 includes three test or target lines, with each target line uniquely corresponding to one of the three antigens. Each target line comprises a plurality of unique species-specific antibody immobilized on the membrane 154, separate from and spaced from other target lines on result module 160. In one aspect, a first target line includes a conjugate of myoglobin, a second target line includes a conjugate of troponin, and a third target line includes a conjugate of cortisol. It is understood that other or additional antigen molecules that are indicative to a cardiac condition of a patient can be used as one of the three or more target lines of test component 166.

Control component 168 of lateral flow device 150 is spaced apart from, and positioned downstream, from test component 166. Control component 168 captures any tagged antigen particles (AT), as well as any unbound tags 164, complex particles that are flowing along lateral flow device 150 but were not captured by target line. In one embodiment, control component 168 comprises an immobile line of anti-species antibody, which causes tagged antigen particles or tags 164 to bind to the control line. When a substantial number of tags 164 and/or tagged antigen particles (AT) are captured, control line is readily visible, thereby indicating that a sufficient volume of the sample has flowed along the lateral flow device to consider the results shown at test component as a valid result. A control line is a mechanism that insures the effectiveness of the lateral flow device to move a sufficient amount of sample past the target line to insure the results at the target line are not falsely based on insufficient volumes of the sample flowing along the lateral flow device.

In one embodiment, test component 166 and/or control component 168 comprises a pattern other than a line or band, such as a word, symbol, curved shape etc., including but not limited to, one of the patterns, orientations, or combinations of patterns shown and described in association with FIG. 5.

Finally, in one embodiment, each of the biosubstances and/or materials comprising results module 158, including but not limited to, test component 166 and/or control component 168, with their accompanying species-specific antibodies and anti-species specific antibodies, are printable onto membrane 154 via jetting assembly 12 (see FIG. 1 and accompanying text) in substantially the same manner as previously described in association with FIGS. 1-2.

Waste module 162 of lateral flow device 150 comprises a collecting area that collects excess sample fluid that has flowed beyond the results module 160 without being captured by test component 166 or control component 168. In one embodiment, waste module 162 comprises, among other things, a protein blocker substance, such as BSA, to insure that no visible lines appear in the waste module 162 to prevent confusion with a visible result at test component 166 and/or visible indication at control component 168 in results module 160.

In one embodiment, jetting assembly 12 of dispensing system 10 enables depositing (i.e., printing) each of modules 156-162, and their components, onto membrane 154 from respectively separate reservoirs of fluid supply assembly 14.

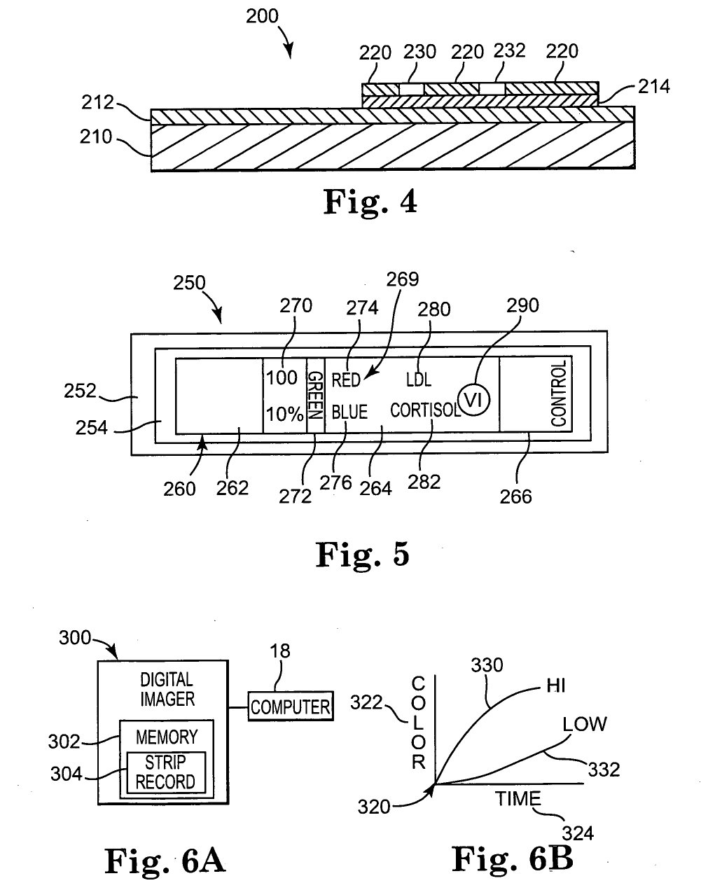

FIG. 4 is a sectional view of a lateral flow device 200, according to one embodiment of the invention. As shown in FIG. 4, lateral flow device 200 comprises carrier 210, membrane 212, pad 214, test region 230, control region 232, and blocking module 220. Carrier 210, membrane 212, test region 230, and control region 232 of lateral flow device 200 have substantially the same features and attributes as corresponding elements of lateral flow device 150 (shown in FIG. 30, namely, carrier 152, membrane 154, and test component and control component of results module 158. Blocking module 220 has substantially the same features and attributes as waste module 162 of lateral flow device 150 (FIG. 3).

In one embodiment, one or more of these modules 156-162 is formed separately from each other by being deposited in layers, contemporaneously or sequentially, via jetting assembly 12. In one aspect, a first orifice plate (FIG. 1) is used to deposit materials to form test region 230 while a second orifice plate is used to deposit materials to form control region 232. Individual reservoirs of the respective fluid arrays 60, 62, 64 of fluid supply assembly (FIG. 1) supply the one or more orifice plates (e.g. first and second orifice plate) for jetting to form the respective modules.

Accordingly, in embodiments in which modules 156-162 are formed in relatively thin layers, such as having a thickness on the order of micrometers or millimeters, several features are realized. First, lateral flow device 150 can be built faster than with conventional manufacturing processes. Second, a thinner module 160 causes a change in the optical density of module 160 to improve the accuracy of a digital imager (e.g. scanner) when quantitatively reading the lateral flow device (to determine the results of the test for the antigen), as later described and illustrated in association with FIG. 6.

FIG. 5 is a plan view of a lateral flow device 250, according to one embodiment of the invention. As shown in FIG. 5, lateral flow device 250 comprises carrier 252, pad 254, lateral flow module 260 including sample portion 262, results portion 264, and waste portion 266. Results portion 264 comprises an array 269 of indicators, including but not limited to, numerical indicators 270 (e.g., 100 and/or 10%), color band indicator 272 (e.g., green or other colors), first color word indicator 274 (e.g., red or other colors), second color word indicator 276 (e.g., blue or other colors), first analyte word indicator 280 (e.g., LDL or another analyte), and second analyte word indicator 282 (e.g., cortisol or another analyte), and symbol indicator 290 (e.g., VI or another graphical pattern). A given lateral flow device can include all of these indicators, only one of these indicators, or several of these indicators, as well as other indicators.

As shown in FIG. 5, in one embodiment first analyte word indicator 280 and/or second word analyte indicator 282 is deposited in the pattern shown via jetting assembly (FIG. 1) and includes anti-species antibody molecules to enable the printed patterns to act as control lines, or control components to indicate successful flow of the sample along the lateral flow device, and simultaneously reveal the name of the antigen for which the test is being performed.

As shown in FIG. 5, in one embodiment numerical indicator 270 is deposited in the pattern shown, via jetting assembly 12 (shown in FIG. 1) during manufacture of lateral flow device 250, and includes species-specific antibody molecules to enable the printed patterns to act as test lines, or test components for binding with the tagged antigen particle (AT) (shown in FIG. 3) to indicate the presence or absence, depending upon the type of assay test, of the antigen, and simultaneously reveal a quantitative parameter (e.g., quantity, concentration or percentage) of the antigen corresponding to activation of the target line or target component. In one aspect, as shown in FIG. 4, more than one numeric target indicator can be presented.

Moreover, in other embodiments, word indicators 280 and 282 are presented as test lines of results portion 264 while numeric indicators 270 are presented as control lines of results portion 264. In this instance, the word indicators 280, 282 would be deposited in a quantity that corresponds to the numeric value of the numeric indicators so that activation of the word indicators in the test line corresponds quantitively with the numeric value shown in the control line.

In one embodiment, when visibly present on test portion 264, color band indicator 272 indicates either a positive result or a negative result, depending upon the design of the assay.

In another embodiment, color word indicators 274, 276 represents three types of information. First, when color word indicator 274,276 is a given color, such as red, the red color can indicate a warning or other qualitative parameter. Second, the word or symbol in the color word indicator 272, 274 provides another type of information as to which antigen was detected, a quantity, and a positive or negative indication, based on the content of the word or symbol. Third, the color word indicator 274, 276 also provides a third type of information in that the visible presence or absence of the indicator on the results module indicates a parameter about the test, such as the presence or absence, or vice versa of an antigen or analyte in the sample.

In one embodiment, indicators 274-282 reveal that the result information of the assay test is displayable in orientations generally parallel to the flow of the sample along the lateral flow device (e.g., generally parallel to a longitudinal axis of the device), thereby making the results of the test more readable.

As shown in FIG. 5, these indicators (270-290) provide considerably more information about one or more antigens tested than information provided by conventional lateral flow through (LFT) sensors. This significant increase in the amount and different types of information revealed in the test results in embodiment of the invention are enabled by the ability of dispensing system 10 to precisely and accurately deposit the biosubstances and/or materials in known quantities and many different positions and orientations unseen in conventional lateral flow through (LFT) sensors, which are made through ordinary coating or dipping techniques. In one aspect, the volume precision, accuracy and positioning of dispensing system (FIGS. 1 and 2) enable printing words, symbols, patterns that are readable with the human eye.

Finally, embodiments of the invention as illustrated and described in association with FIG. 5 reveal the action of dispensing system 10 creating simultaneous formation (from a deposited biosubstance or material) of an assay reaction mechanism of the lateral flow device (e.g., a test line for interaction of a tagged antigen particle and a species-specific antibody) and a set of quantitative indicators (by numbers, word, pattern or color) that appear directly on the test portion. Accordingly, embodiments of the invention enable quantitative results reporting, independent of calorimetric readers, caused by the assay reaction mechanism itself, while also not excluding the additional use of colorimetric readers or other readers.

FIG. 6A is a block diagram of a digital imager 300 for use in reading a lateral flow device, according to one embodiment of the invention. As shown in FIG. 6A, digital imager 300 comprises a scanner or other device configured to capture digital images of an object or surface, such as a lateral flow device, through optical scanning or other means. Digital imager 300 includes, among other things, memory 302 for storing strip record(s) 304. This feature enables a record of lateral flow device to be stored for current or later analysis, as well as archival of the test results of a lateral flow device. Unlike the visible results on the surface of a conventional lateral flow device, which deteriorate over time, a digital record of the visible results of a lateral flow device can be maintained indefinitely. This digital record can be used as a permanent medical record, a legal record, research data, or other purposes.

In another aspect, digital imager 300 enables determining test results on a quantitative basis by detecting a volume of color within an image field, an intensity of color, and/or rate of color change over time as the test result is developing. In detecting a rate of color change, as the assay test is being performed, a curve is fit of the rate of color change to enable predicting the full curve of color change over time without waiting for the full time required to finish the immunoassay test. In one aspect, as shown in FIG. 6B, a graph 320 of color change over time includes a color axis 322 and a time axis 324 with representative curves plotted, respectively illustrating a high concentration of an analyte in curve 330 and a low concentration of an analyte in curve 332. In this way, nearly instantaneous results can be determined and reported for the immunoassay test of the lateral flow device.

In another embodiment, after the immunoassay test has completely run its course, digital imager 300 enables quantifying the test result based on the intensity and/or volume of color visible on results module or portion of the lateral flow device wherein the digital imager 300 (with or without computer 18) quantifies, on a pixel-by-pixel basis, the amount or concentration of an analyte based on the imaged color on the surface of the test result module of the lateral flow device.

These embodiments of digital imager 300 overcome many errors associated with humans attempting to determine the presence or absence of a line, the relative color of a line, as well as human memory in remembering whether a present line indicates a negative result or a positive result. Use of digital imager alleviates these issues by accurately reporting the result based on the image obtained from the lateral flow device.

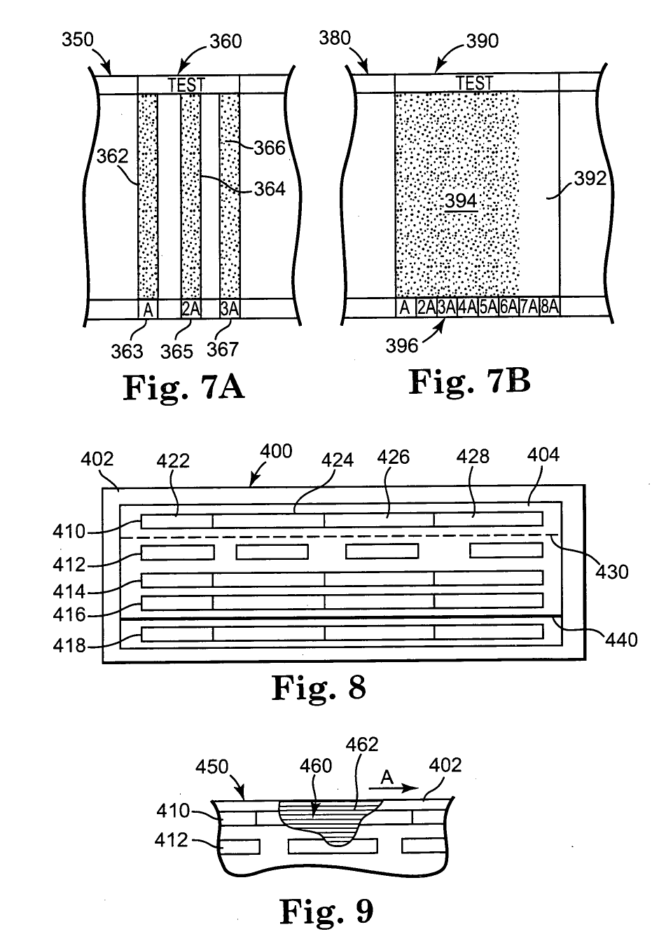

FIG. 7A is a partial plan view of a lateral flow device, according to an embodiment of the invention. As shown in FIG. 7A, lateral flow device 350 comprises, among other things, test module 360 including first test band 362 with corresponding information indicator 363, second test band 364 with corresponding information indicator 365, and third test band 366 with corresponding information indicator 367. In one embodiment, the three test bands correspond to multiples (e.g., A, 2A, 3A) of a given quantity (e.g. value A) of the volume or concentration of an analyte. Accordingly, in this embodiment, each informational indicator indicates a quantity of the analyte. In other embodiments, each informational indicator can use symbols or colors to represent different quantity information about the respective test bands.

The test bands are configured so that as the sample progresses across test field 360, the number of test bands that achieve color reveal the quantity of antigen detected. In other words, each test band exhibits same quantity of analyte so the total quantity in the sample is revealed by adding up the number of activated test bands.

In some embodiments, some test bands have a higher concentration or higher volume of target species antibody (e.g., twice or three times as much as another band) so that achieving color in successive test bands reveals more than simple multiples of activation of a single test band.

In one aspect, an analyte such as one of the cardiac panel markers is represented by an array of test lines, with each successive test line (362, 364, 366) quantitatively corresponding to a greater percentage of the cardiac antigen in the sample. Since jetting assembly 12 is capable of jetting the species-specific antibody onto results module 360 in precise, measurable quantities, and each “activated” target line corresponds to a selected quantity, one can determine a quantity of antigen in the sample by the number of target lines (362-366) that are activated. This feature enables a test result that is quantitative rather than an all-or-none qualitative conventional test.

In another embodiment, the three (or more) test lines 362, 364, 366 do not represent different quantitative levels of the same antigen, but instead each line represents a different antigen so that all three unique antigens, such as separate cardiac panel markers are revealed on a single lateral flow device.

In one embodiment, the information indicators 363, 365, 367 are printed with conventional inks along the lateral flow device on a surface independent from, but adjacent to, the assay membrane and its modules, with a position of each of these indicators 363, 365, 367 corresponding to a respective test line or component (e.g. 362, 364, and 366 respectively). In another embodiment, information indicators 363, 365, and 367 are deposited as part of each test line (362, 364, and 366) and become visible only when the test line (corresponding to the quantity expressed by the numeral of the indicator) is activated during the immunoassay test.

FIG. 7B is a partial plan view of a lateral flow device 380, according to an embodiment of the invention. As shown in FIG. 7B, lateral flow device 380 comprises, among other things, test module 390 including first field 392 with test quantity 394 and corresponding information indicator array 396. Test quantity 394 is a variable parameter that is visible in proportion over the first field 392 relative to the volume of antigen detected. Information indicator array 396 extends generally parallel to test field 392 and includes markings corresponding to a volume or amount of an analyte represented by relative progress of colored portion 394 across test field 392. In one embodiment, the measured quantity is represented by values in multiples of A (e.g., A, 2A, 3A, 4A, 5A, etc). In other embodiment, different quantitative relationships can be represented by successive markers of the array 396 (e.g. non-linear, exponential) enabled by the capacity of dispensing system 10 to deposit test lines, regions, patterns in corresponding non-linear or exponential gradients across test field 392.

In one embodiment, the total volume of test quantity 394 and/or intensity of color and/or volume of color of test quantity 394 can be obtained by digital imager 300 to overcome human error in interpreting the results of the test. This feature is particularly helpful when test lines are deposited on test field 392 in non-linear or exponential gradients, as the human eye would be incapable of accurately quantifying a volume and/or intensity of color expressed by test quantity 394 on first test field 392.

In another embodiment, test module 390 includes more than one test field. Multiple test fields can correspond to a single analyte or to different analytes, with each separate test field being activated by a different antigen.

In one embodiment, the quantity indicators of quantity indicator array 396 are printed with conventional inks along the lateral flow device on a surface independent from the assay membrane and its modules, with a position of each of these indicators (e.g. A, 2A, etc corresponding to a respective position along a gradient of test field 392). In another embodiment, quantity indicators of array 396 are deposited as part of test field 393 and become visible only when the corresponding test quantity 394 along the gradient of test field 392 is activated during the immunoassay test.

FIG. 8 is a plan view of a lateral flow device 400. As shown in FIG. 8, a single lateral flow device 400 comprises, among other things, a carrier 402, membrane 404, and multiple lateral flow modules (410, 412, 414, 416, and 418) arranged generally parallel to each other in a side-by-side relationship. Each lateral flow module (410-418) is functionally independent of adjacent lateral flow modules, regarding the test result obtained, yet related in that a single membrane 404 supports wicking and/or migration of a single fluid sample across multiple modules of lateral flow device 400. In one embodiment, each lateral flow module includes components sufficient to perform its own immunoassay test independent of the other lateral flow modules (410-418), with components such as a sample portion 422, a reagent portion 424, a results portion 426 (with a test line and a control line), and a waste portion 428.

Various mechanisms enable maintaining independence of testing between adjacent lateral flow modules (410-418). In one embodiment, lateral flow device 400 includes a chemical barrier 430 such as a hydro-responsive material (e.g., hydrophilic or hydrophobic substance) deposited, via dispensing system 10 from carrier module 52 of fluid supply assembly 14, between adjacent modules (e.g. module 410 and module 412). In another embodiment, lateral flow device 400 includes a physical barrier 440, such as a plastic wall or other substantially impenetrable material separating adjacent modules (e.g., module 416 and module 418). These barriers can be placed between each set of adjacent modules, or only one or two specific sets of adjacent modules.

FIG. 9 illustrates a lateral flow device 450, according to an embodiment of the invention. In one embodiment, lateral flow device 450 has substantially the same features and attributes as lateral flow device 400, except instead of having barriers on top surface of lateral flow device 450 between adjacent modules as shown in FIG. 8, lateral flow device 450 includes an array 460 of unidirectional capillaries 462. As shown in FIG. 9, unidirectional capillaries 462 of array 460 extend generally parallel to a direction of flow of lateral flow device 450 for directing flow of fluids within and surrounding a certain module to be maintained separately from flow of fluids within and surrounding an adjacent module.

FIG. 10 is a block diagram of a control monitor 500, according to an embodiment of the invention. Control monitor 500 represents a component of dispensing system 10 or an associated computer 18, such as portion of a controller or driver, for directing dispensing system 10 to deploy the embodiments described and illustrated in association with FIGS. 1-9. Control monitor 500, including all of its monitors, represents underlying executable modules in software, firmware, hardware that enable the identified functions and parameters in control monitor 500 as well as their display and activation via a user interface, such as user interface 24 in FIG. 1.

As shown in FIG. 10, control monitor 500 comprises a module formation monitor 502, an indicator monitor 504, and an image reader monitor 506.

Module formation monitor 502 enables selections and creation of various modules (e.g. sample module, reagent module, results module, etc.) of a lateral flow device via dispensing system 10. Module formation monitor 502 comprises size parameter 510, depth parameter 512, volume parameter 514, dimension parameter 516, position parameter 518, layer parameter 520, orientation parameter 522, functional module parameter 526, functional biounit parameter 528, parallel parameter 530, perpendicular parameter 532, multiple assay module parameter 534, barrier parameter 535, line parameter 536, gradient parameter 537, antigen parameter 538, antibody parameter 539, tag parameters 540 including color parameter 542, marker type parameter 544, and particle parameter 546, as well as program parameter 550 and database parameter 552.

Size parameter 510 and depth parameter 512 of module formation monitor 502 respectively control selecting and forming a size and a depth of one or more modules of a lateral flow device. Volume parameter 514 of module formation monitor 502 controls selecting and forming a volume one or more modules (e.g. reagent module, results module, waste module, etc.), including selection and control of a volume of each biosubstance and/or other materials of forming each module, of a lateral flow device.

Dimension parameter 516, position parameter 518, and orientation parameter 522 of module formation monitor 502 respectively control selecting and forming a dimension (e.g., width, length, shape, etc.), a position (e.g., middle, edge, x-y coordinates, etc.), and an orientation (e.g., parallel, perpendicular, angled, etc.) of one or more modules (e.g., results, reagent, etc.) of a lateral flow device.

Layer parameter 520 of module formation monitor 502, when activated, directs how many, and the depth of each layer deposited via dispensing system 10. Functional module parameter 526 directs more than one biosubstance and/or other material to be deposited contemporaneously or sequentially, as one or more separate modules to enable dispensing system 10 to form entire modules on the lateral flow device.

Functional biounit parameter 528 of module formation monitor 502 directs more than one biosubstance and/or other material to be deposited contemporaneously or sequentially at the same location to form a single biounit having a predetermined immunoassay function on the lateral flow device. Multiple functional biounits can be printed in adjacent locations, in patterns, spaced apart form each other, as well as in sheets for forming layers. The different biosubstances and/or other materials are supplied from arrays 60-64 of fluid supply assembly 14 and jetted from jetting assembly 12 via one or more orifice plates via nozzles 36 and/or 38, as previously described and illustrated in association with FIGS. 1-9.

Parallel parameter 530 and perpendicular parameter 532 of module formation monitor 502 respectively control selecting and forming whether a module (e.g., a results module, a reagent module, etc.) and/or component (e.g., a test line, a control line, informational indicators, etc.) of a module is deposited, respectively, generally parallel to or generally perpendicular to, a flow direction of the lateral flow device.

Multiple assay module parameter 534 of module formation monitor 502 directs dispensing system 10 to select and form independent multiple assay modules in a generally parallel orientation, side-by-side, on a single lateral flow device. Barrier parameter 535 controls whether or not barriers are formed between adjacent (side-by-side) assay modules, how many barriers are formed, and what type of material is used to form the barriers.

Line parameter 536 of module formation monitor 502 directs dispensing system 10 to control the width, position, and concentration (e.g., concentration of biosubstances such as species-specific antibody) of a test line and/or of a control line of a results module of a lateral flow device, as well as to control the number of test lines, and spacing between adjacent test lines. Gradient parameter 537 of module formation monitor 502 direct dispensing system 10 to control the width and position of a gradient test field, as well as the distribution (e.g., uniform, increasing, decreasing, etc.) of the concentration of biosubstances (e.g., species-specific antibodies) within the gradient test field.

Antigen parameter 538 and antibody parameter 539 of module formation monitor 502 direct dispensing system 10 to select and control which antigens and antibodies, respectively, are deposited on a lateral flow device, in accordance with other parameters selected by user interface, as the number of different antigens and antibodies, respectively that are deposited.

Tag parameters 540 of module formation monitor 502 directs dispensing system 10 to select and control factors affecting equipping an antigen molecule in a sample pad and/or reagent pad for flowable movement along lateral flow device with a readable color. Color parameter 542 of tag parameters selects one or more colors for association with one or more antigens, or one or more types of results for an antigen. Marker type parameter 544 selects whether the color is marked by a metal such as gold, or by a colored latex bead. Particle parameter 546 selects which type of flowable particle, such as a latex bead, or microsphere that is attached to the antigen molecule.

Program parameter 550 of module formation monitor 502 comprises control over selectable programs, having a complete set of parameters of module formation monitor 502 already selected to build a lateral flow device according to previously programmed selections or instructions. Database parameter 552 selects the source (e.g., computer 18, or another external computer, server, storage media, etc.) for obtaining and supplying the previously created programs selected by program parameter 550.

Indicator monitor 504 of control monitor 500 is configured to direct dispensing system 10 to deposit biosubstances and/or other materials onto a lateral flow device for visually indicating a parameter (e.g., name of analyte, quantity detected, positive result, etc.) of an immunoassay test, in human readable and/or machine readable forms. Indicator monitor 504 comprises pattern parameter 570, word parameter 572, symbol parameter 574, numeral parameter 576, color parameter 578, orientation parameter 580 including selectors such as parallel, angled, perpendicular.

Pattern parameter 570 selects any discernible pattern carrying information about the immunoassay test. Word parameter 572, symbol parameter 574, and numeral parameter 576, select which and how many words, symbols, and numerals, respectively, are incorporated into a test component or control component of a results module of a lateral flow device for expressing the results or other information about an immunoassay test. Likewise, color parameter 578 selects which and how many colors are incorporated into a test component or control component of a results module of a lateral flow device for expressing the results or other information about an immunoassay test. Orientation parameter 580 selects one or more orientations (e.g., parallel, angled, perpendicular) of words, symbols, and patterns relative to a longitudinal axis of a lateral flow device for expressing the results or other information about an immunoassay test.

Image reader monitor 506 of control monitor 500 comprises functions and parameters to direct operation of a digital imager, such as digital imager 300, and manipulation of digital images obtained by digital imager 300 described and illustrated in association with FIG. 6A. Image reader monitor 506 comprises reading parameters 600 and record parameters 602, which can be independent or interdependent aspects of image reader monitor 506. Reading parameters 600 directs what types of information digital imager 300 obtains from a lateral flow device, including but not limited to, intensity parameter 610, volume parameter 612, color selector parameter 614, and curve fit parameter 616. Intensity parameter 610 controls activation of reading an intensity of color of a test result while volume parameter controls reading a result module for a volume of color. Color selector parameter 614 selects which color or colors that digital imager 300 will focus on in reading a lateral flow device. Curve fit parameter 616 determines whether digital imager 300 will be used to obtain a rapid result by fitting a curve of a color change over time for a developing result module of a lateral flow device.

Record parameters 602 directs how a digital image of a lateral flow device is handled, evaluated, and/or reported. Record parameter 602 comprises recording parameter 620, storage parameter 622, database parameter 624, and reference parameter 626, display parameter 628, and report parameter 630. Recording parameter 620 selects and controls whether digital imager 300 will make a recording of the lateral flow device, and storage parameter selects and controls whether and when a digital image of a lateral flow device will be stored as a permanent record by digital imager 300 for posterity or for transfer to another medical record storage device. Database parameter 628 selects and controls whether a digital image of a lateral flow device is stored in a database or compared with digital images in a database. Reference parameter 626 selects and controls whether a digital image of a lateral flow device is evaluated for its results based on reference criteria about the antigen or other factors. Display parameter 638 controls whether the actual digital image of a lateral flow device, and/or what type of information read from that digital image, will be displayed on user interface 500 or another display mechanism. Report parameter 640 controls what detailed information about an immunoassay test imaged by digital imager 300 will be reported, with a user capable of selecting any of the parameter of user interface 500 for inclusion in a report about an imaged lateral flow device. This report information can be stored as a record along with the digital image of the lateral flow device.

Embodiments of the present invention are directed to enabling faster production of lateral flow devices, as well as producing more readily quantifiable results. Via use of a drop-on-demand dispensing system, lateral flow devices are transformed into devices carrying much more quantifiable information than conventional crude qualitative results in conventional lateral flow through sensors. Each lateral flow device can be custom made, with rapid design and formation of the lateral flow devices, thereby enabling lateral flow devices to be used in quickly evolving health crises, such as a sudden acute respiratory syndrome (SARS) epidemic. The results of the lateral flow devices are more readily readable by human eyes, as well as more readily readable by machine readers. Embodiments of the invention transform a lateral flow device from a crude initial disposable test, into a sophisticated evaluation of an antigen sample that will be storable as a permanent record.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A dispenser for building lateral flow immunoassay devices comprising:
a supply assembly comprising an antibody component including a plurality of reservoirs to supply at least one member of a group comprising:
a species array of at least one species-specific antibody material;
an anti-species array of at least one first anti-species antibody material; and
a label-antibody array of at least one of a tag material and a second anti-species antibody material; and
a jetting assembly in communication with the supply assembly to enable dispensing the materials of the species array, the anti-species array, and the label-antibody array from the supply assembly onto a substrate.

2. The dispenser of claim 1 wherein the supply assembly comprises a carrier component including a reservoir containing at least one liquid material configured to facilitate dispensing the antibody materials.

3. The dispenser of claim 1 wherein the supply assembly comprises a support component configured to contain at least one member of a group comprising a surfactant, an adjuvant, a barrier material, a screening material, and a protein blocker.

4. The dispenser of claim 1 and further comprising:
a controller in electrical communication with the jetting assembly and the fluid supply assembly to control operation of the dispensing system.

5. The dispenser of claim 4 wherein the controller comprises:
a module formation monitor configured to enable dispensing the materials to form modules of a lateral flow immunoassay device wherein the module formation monitor includes a plurality of parameters corresponding to different aspects of the lateral flow immunoassay device, the parameters including at least one member of a group comprising:
a size parameter;
a depth parameter;
a layer parameter;
a volume parameter;
a dimension parameter;
an orientation parameter; and
a position parameter.

6. The dispenser of claim 4 wherein the controller comprises:
a module formation monitor configured to enable dispensing the materials to form modules of a lateral flow immunoassay device wherein the module formation monitor includes at least one parameter corresponding to an aspect of the lateral flow immunoassay device, the at least one parameter including a functional biounit parameter.

7. The dispenser of claim 4 wherein the controller comprises:
a module formation monitor configured to enable dispensing the materials to form modules of a lateral flow immunoassay device wherein the module formation monitor includes a plurality of parameters corresponding to different aspects of the lateral flow immunoassay device, the parameters including at least one member of a group comprising:
a side-by-side independent assay parameter; and
an adjacent assay barrier parameter.

8. The dispenser of claim 4 wherein the controller comprises:
a module formation monitor configured to enable dispensing the materials to form modules of a lateral flow immunoassay device wherein the module formation monitor includes a plurality of parameters corresponding to different aspects of the lateral flow immunoassay device, the parameters including at least one member of a group comprising:
an antigen selector parameter;
an antibody selector parameter;
a result line parameter;
a result gradient parameter; and
a tag selector parameter including a color parameter, a marker parameter, and a flowable particle parameter.

9. The dispenser of claim 4 wherein the controller comprises a control monitor, accessible via an user interface, configured to display and enable activation of at least one member of a group comprising a module formation monitor, an indicator monitor, and an image reader monitor of the dispenser.

10. The dispenser of claim 1 and further comprising:
an indicator monitor configured to control depositing the materials onto the substrate wherein the indicator monitor includes a plurality of parameters corresponding to visual indicators of the lateral flow immunoassay device, the parameters including at least one member of a group comprising:
a result pattern parameter;
a word parameter;
a symbol parameter;
a numeral parameter;
a color parameter; and
an orientation parameter.

11. The dispenser of claim 1 and further comprising an image reader configured to optically read a lateral flow device wherein the image reader comprises a reading monitor for optically detecting different aspects of the lateral flow device corresponding to an array of parameters including at least one member of a group comprising:
an intensity parameter;
a volume parameter;
a color parameter; and
a curve parameter.

12. The dispenser of claim 9 wherein the image reader comprises a record monitor for optically recording and evaluating different aspects of the lateral flow device including at least one member of a group comprising:
a recording parameter;
a storage parameter;
a result database parameter;
a test reference parameter;
a display parameter; and
a report parameter.

13. The dispenser of claim 1 further comprising a handling station enabling three-dimensional positioning of the jetting assembly relative to a substrate to enable depositing the materials onto the substrate.

14. A dispenser for creating a lateral flow device comprising:
means for maintaining an array of biosubstances adapted to form a labeling reaction portion, a test capture reaction portion, and a control reaction portion;
means for separately depositing at least one biosubstance of the array of biosubstances from the means for maintaining onto a membrane to form the labeling reaction portion, the test capture portion and the control reaction portion of the lateral flow device; and
means for controlling the means for maintaining and the means for separately dispensing to enable customizable formation of each lateral flow device.

15. The dispenser of claim 14 wherein the means for maintaining comprises a fluid supply assembly including an array of fluid reservoirs including a first array of fluid reservoirs configured to contain label reaction materials, a second array of fluid reservoirs configured to contain test capture reaction materials, and a third array of fluid reservoirs configured to contain control reaction materials.

16. The dispenser of claim 15 wherein the means for separately depositing comprises:
a drop-on-demand fluid ejection device including at least one orifice plate and an array of nozzles to enable separate dispensing of different biosubstances through a respective different nozzle with the dispensing performed at least one of simultaneously and sequentially.

17. The dispenser of claim 16 wherein the means for controlling comprises:
a parameter user interface configured to enable user selection of at least one parameter of the label reaction portion, of the test capture reaction portion, and of the control reaction portion, the at least one parameter including at least one member of a group comprising;
a depth parameter;
a layer parameter;
a volume parameter;
a dimension parameter;
an orientation parameter; and
a position parameter.

18. A method of dispensing biosubstances for a lateral flow assay device, the method comprising:
supplying an array of biosubstances, including a species-specific antibody material, an anti-species antibody material, a labeled anti-species antibody material, separately in an array of fluid reservoirs; and
jetting the biosubstances, via a fluid ejection device, separately from the array of fluid reservoirs onto a membrane, including jetting the labeled anti-species antibody material to form a reagent module, the species specific antibody material to form a results module, and an anti-species antibody material to form a control module of the lateral flow assay device.

19. The method of claim 18 wherein jetting the biosubstances comprises:
separately controlling the jetting, via a user interface, of at least one of an orientation, layering, volume, pattern, and position of each biosubstance on the membrane to enable formation of each of the reagent module, the results module and the control module.

20. The method of claim 19 wherein jetting the biosubstances comprises jetting each different biosubstance through a separate nozzle of the fluid ejection device to enable simultaneous depositing of the different biosubstances on the membrane.

21. The method of claim 19 wherein jetting the biosubstances via a fluid ejection device comprises is performed via a drop-on-demand fluid ejection device.

22. The lateral flow assay device manufactured according to the method of claim 18.

23. A system for lateral flow device operations comprising:
a supply assembly comprising an antibody component including a plurality of reservoirs to supply:
a species array of at least one species-specific antibody material;
an anti-species array of at least one first anti-species antibody material; and
a label-antibody array of at least one of a tag material and a second anti-species antibody material; and
a jetting assembly in communication with the supply assembly to enable dispensing the materials of the species array, the anti-species array, and the label-antibody array from the supply assembly onto a substrate; and
a controller in electrical communication with the jetting assembly and the fluid supply assembly to control operation of the dispensing system, the controller including:
a module formation monitor configured to enable dispensing of the materials to form modules of a lateral flow immunoassay device wherein the module formation monitor includes a plurality of parameters corresponding to different aspects of the lateral flow immunoassay device; and
an image reader monitor configured to direct obtaining an image of a lateral flow device wherein the image reader monitor comprises a reading monitor for optically detecting different aspects of the lateral flow device and a recording monitor for storing and evaluating the image.
